(19) United States
(12) Patent Application Publication
(10) Pub. No.: US 2015/0241411 A1
(43) Pub. Date: Aug. 27, 2015

(54) IDENTIFICATION OF ENZYME ACTIVITY THROUGH DETERMINATION OF ITS LOCALIZATION

(71) Applicant: International Business Machines Corporation, Armonk, NY (US)

(72) Inventor: Pablo Meyer Rojas, Yorktown Heights, NY (US)

(21) Appl. No.: 14/583,841

(22) Filed: Dec. 29, 2014

Related U.S. Application Data
(60) Provisional application No. 61/944,200, filed on Feb. 25, 2014.

(51) Int. Cl. G01N 33/50; C12Q 1/48; G01N 33/58; C12Q 1/02

(52) U.S. Cl. CPC G01N 33/5035; C12Q 1/025; C12Q 1/48; G01N 33/5041; G01N 33/582; G01N 2500/10; G01N 2333/91171

(57) ABSTRACT

A method of determining enzyme activity and identifying and classifying cellular targets, enzymatic pathways, and enzymatic agents involved in regulating metabolism in order to treat pathophysiological disorders. Monitoring enzyme activity is performed via a label-free bio cellular assay or fluorescence imaging. The identified and classified agents are used, together with a therapeutic agent, in the treatment of various metabolism-related diseases.

### PRIORITY

This application is a non-provisional of and claims priority from U.S. Patent Application Ser. No. 61/944,200, filed on Feb. 25, 2014, entitled “IDENTIFICATION OF ENZYME ACTIVITY THROUGH DETERMINATION OF ITS LOCALIZATION”, the entire contents of which are incorporated herein by reference.

### BACKGROUND

The present invention generally relates to methods of determining enzyme activity involved in regulating metabolism. More particularly, the present invention relates to identifying cellular targets, enzymatic pathways, and enzymatic agents in order to treat pathophysiological disorders.

Enzymes are responsible for the metabolic needs, capabilities, and possibilities of living organisms. Enzymes are required in almost all chemical reactions in a cell and their activity determines which metabolic pathways can occur. While enzymes play a very important role in sustaining life, the malfunction of a single enzyme, through a mutation, deletion, etc., can lead to diseases, such as cancer or diabetes. Therefore, it is important to determine which enzymes are active in different disease situations.

Enzyme activity can be measured using either direct or indirect methods. Indirect methods include measuring the synthesis rate of its product or the consumption rate of its substrate as measured by mass spectroscopy, liquid chromatography, or via in vitro chemical assays. Direct methods include magnetic resonance imaging to measure in vivo enzyme activity. However, these methods are cumbersome and cannot be scaled to many enzymes or to different time points.

Accordingly, improved methods for determining enzyme activity are needed, in particular how enzyme localization reflects enzyme activity.

### SUMMARY

One aspect of the present invention provides a method of identifying cellular targets involved in regulating enzyme condensation dynamics which includes the steps of providing a cell that has a cellular target, contacting the cell with a molecule that interacts with the cellular target forming a molecule-contacted cell, exposing the molecule-contacted cell to an enzyme condensation promoting agent, exposing the molecule-contacted cell to an enzyme condensation disrupting agent, monitoring the state of enzyme condensation of the molecule-contacted cell after exposure to the enzyme condensation promoting agent and the enzyme condensation disrupting agent, and determining the molecule's ability to regulate the state of enzyme condensation.

Another aspect of the present invention provides a method of identifying an enzyme condensation dynamics modulating pathway which includes the steps of providing a cell that has a cellular target, contacting the cell and (i) a cellular pathway modulator that interacts with the cellular target and (ii) a cellular target specific ligand forming a cellular pathway modulator and ligand-contacted cell, exposing the cellular pathway modulator and ligand-contacted cell to an enzyme condensation modulating agent, assaying the cell response to the enzyme condensation modulating agent, and determining the cellular pathway modulator's ability to regulate the state of enzyme condensation.

Yet another aspect of the present invention provides a method of identifying agents that regulate enzyme condensation dynamics including the steps of providing a cell, forming a molecule-contacted cell by contacting the cell with a molecule, exposing the molecule-contacted cell with an enzyme condensation promoting agent, exposing the molecule-contacted cell with an enzyme condensation disrupting agent, monitoring the state of enzyme condensation of the molecule-contacted cell after exposure to the enzyme condensation promoting agent and the enzyme condensation disrupting agent, and determining the modulating ability of the molecule on the state of enzyme condensation.

A further aspect of the present invention provides a method of determining the metabolic state of an enzyme through enzyme condensation comprising the steps of providing a cell having a cellular target, attaching a reporter protein to the cellular target by contacting the reporter protein with the cell, exposing the cell with an enzyme condensation promoting agent, exposing the cell with an enzyme condensation disrupting agent, monitoring the state of enzyme condensation of the cell after exposure to the enzyme condensation promoting agent and the enzyme condensation disrupting agent, and determining the state of enzyme condensation.

Yet another aspect of the present invention provides a method of treating a subject having a disease that is pathophysiologically related to enzyme condensation, the method including the step of administering a therapeutically effective amount of an enzyme condensation dynamics modulator for modulating enzyme condensation.

The present invention has many advantages over other methods, such as mass spectroscopy, liquid chromatography, magnetic resonance imaging, etc. The present method can be scaled to many enzymes at a lower cost and it is also more flexible and simpler to implement.

### BRIEF DESCRIPTION OF THE DRAWINGS

These and further features of the present invention will be apparent with reference to the following description and drawing, wherein:

FIG. 1 is a schematic diagram illustrating the organization of enzymes, A to H, in a metabolic pathway, going from diffuse localization in the cytoplasm when the enzymes are not active (left) to a discrete concentration of enzymes when the pathway is active (right); and

FIG. 2 is a diagram of images of enzymes during glycolysis and the TCA cycle, showing both localized enzyme activity (active) and diffuse enzyme activity (not active).

### DETAILED DESCRIPTION OF THE PREFERRED EMBODIMENT

The following detailed description of various preferred embodiments will illustrate the general principles of the present invention with reference to methods of identifying cellular targets involved in enzyme condensation dynamics, enzyme condensation dynamics pathways, and enzyme condensation dynamics modulators, and their use in the treatment of pathophysiological diseases for purposes of clarity and not by way of limitation. The application of the preferred embodiments in other contexts will be apparent to those skilled in the art given the benefit of this disclosure.

Although the enzymatic cascades that underlie cellular biosynthesis are understood, comparatively little is known about the rules that determine their cellular organization. The image of a cell as a “bag of enzymes” has given way to a view where molecules and proteins localize at the right time in the right place in order to perform their necessary functions. However, enzymes involved in the most basic metabolic functions are generally thought to be freely diffusing in the cytoplasm. The existence of large multi-enzyme complexes, as opposed to freely diffusing enzymes, could either be determined by constraints limited to highly specialized reactions or a general mechanism used throughout the cell to achieve a generic metabolic function.

It is a general property of enzymes that globular non-membrane bound enzymes of bacterial and eukaryotic cells concentrate to cytoplasmic foci when active and are diffuse when they are inactive. When a metabolic pathway is active, the first and last enzymes of such biosynthetic pathway condense to precise cytoplasmic foci, but are homogenously distributed during stationary growth, suggesting that the concentration of enzymes to discrete foci occurs when they are most active. Thus, enzyme condensation reflects the activity state of a cell's metabolism, following a pathway specific first-or-last enzyme localization rule and also interconnecting active metabolic pathways. Such an enzyme condensation event is hereby described as a direct way to screen for agents regulating cell metabolism via the regulation of the dynamics of enzyme condensates.

The invention includes determining the metabolic state, i.e., active or inactive, of each kind of enzyme in the organism under study. This is done by determining whether the enzyme is focalized (active) or diffuse (inactive) in the cytoplasm. One way of determining the metabolic state of an enzyme is to attach a fluorescent reporter protein or any other sort of reporter to the carboxyl terminal portion or any other portion of each enzyme and use a method, such as a fluorescent microscope, to determine whether the enzyme is focalized (condensed) or diffuse in the cell cytoplasm.

Reporters of different wavelengths can be attached to different enzymes, allowing one to determine the enzymatic activity of multiple enzymes simultaneously. In other circumstances, enzyme activity can be determined one enzyme at a time.

This method of identifying enzyme activity has many advantages over other methods, such as mass spectroscopy, liquid chromatography, magnetic resonance imaging, etc. The present method can be scaled to many enzymes at a lower cost. It is also more flexible and simpler to implement.

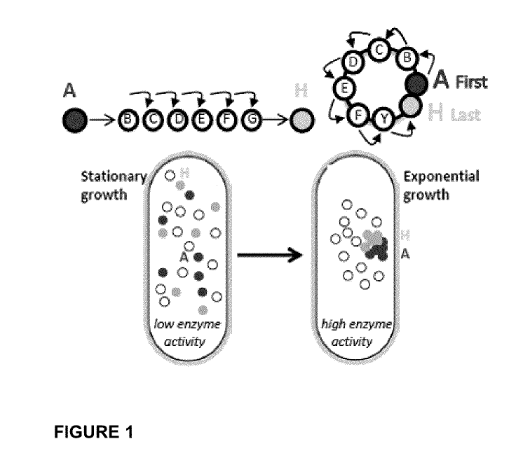

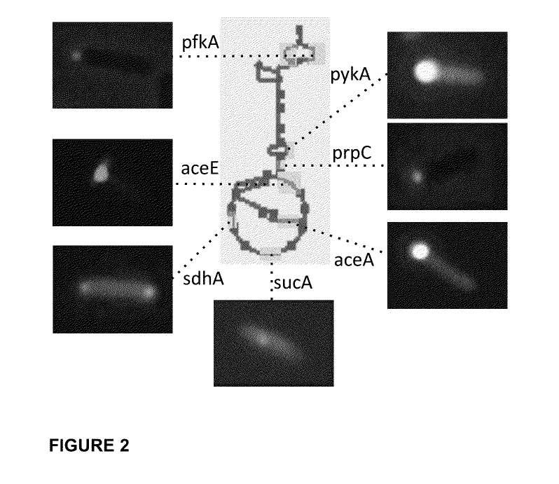

FIGS. 1 and 2 illustrate the methods of identifying cellular targets involved in regulating enzyme condensation dynamics. FIG. 1 shows the condensation of enzymes, wherein during low enzyme activity, the enzymes are diffuse throughout the cell and during high enzyme activity, the enzymes localize to particular foci within the cell. FIG. 2 shows images of localized enzymes from glycolysis and the TCA cycle.

Referring to FIG. 1, a method is shown which include the steps of providing a cell, contacting the cell with a molecule that interacts with the cell forming a molecule-contacted cell, exposing the molecule-contacted cell to an enzyme condensation promoting agent, exposing the molecule-contacted cell to an enzyme condensation disrupting agent, monitoring the state of enzyme condensation of the molecule-contacted cell after exposure to the enzyme condensation promoting agent and the enzyme condensation disrupting agent, and determining the molecule's ability to modulate the state of enzyme condensation. The step of monitoring the state of enzyme condensation after being exposed to enzyme condensation promoting and disrupting agents can be done through the use of fluorescence imaging of an enzyme that is part of the enzyme condensation complex or through the use of a label-free biosensor-based cellular assay.

In another example, FIG. 2 illustrates the condensation of enzymes during glycolysis and the TCA cycle. In particular, it illustrates a method of identifying cellular targets involved in regulating enzyme condensation dynamics, which includes the steps of providing a cell that has a cellular target. The cell can be, but is not limited to a HeLa cell. The cell is then contacted with a molecule that interacts with the cellular target forming a molecule-contacted cell. The molecule-contacted cell is then exposed sequentially to an enzyme condensation promoting agent and an enzyme condensation disrupting agent. After exposure to the promoting and disrupting agents, the state of enzyme condensation is then monitored in the molecule-contacted cell to determine the modulating ability of the molecule on the state of enzyme condensation. As in FIG. 1, the step of monitoring the state of enzyme condensation after being exposed to enzyme condensation promoting and disrupting agents can be done through the use of fluorescence imaging of an enzyme that is part of the enzyme condensation complex or through the use of a label-free biosensor-based cellular assay.

The molecules that interacts with the cellular target include hormones, signaling effectors, antibodies, antibiotics (such as ampicillin, vancomycin, fosfomycin, erythromicin), bacterial products (such as rapamycin, tryptone, stauromycin, leptomycin-B), plant derived products, synthetic chemicals (e.g., IPTG) or chemical products (e.g., NACL, d-glucose, CaCl₂ , MgCl₂,CuCl₂, ZnCl₂, MnSO₄, MgCl₂, (NH₄)₂SO₄, Na₂MoO₄, CoCl₂, and Ca(NO₃)₂), amino acids (such as 1-glutamic acid, 1-leucine, 1-valine, 1-threonine, 1-methionine, 1-histidine) and carbohydrates and carboxylic acids.

The enzyme condensation promoting agent can be, but is not limited to, a hormone (insulin), a signaling effector (EGF), chemical products (glucose), and amino acids (1-methionine). In some circumstances, the enzyme condensation promoting agent can contact the cell before the enzyme condensation disrupting agent, while in others, the enzyme condensation disrupting agent contacts the cell before the enzyme condensation promoting agent. In either case, the time between contact of the promoting agent and the disrupting agent can be, but is not limited to, 1, 5, 10, 15, 30, or 60 minutes. The time between cell contacts of the agents can be, but is not limited to 1, 2, 3, 4, 5, 10, 12, 18, or 24 hours.

The enzyme condensation disrupting agent can be, but is not limited to, an antibody (anti-her2), antibiotics (fosfomycin), and bacterial products (rapamycin).

The molecule's ability to affect the dynamics of the state of enzyme condensation, i.e., localized or diffuse, demonstrates that the cellular target of the molecule plays a role in regulating enzyme condensation dynamics. The molecule can be, but is not limited to, an agonist.

Another way to identify enzyme activity is via enzyme condensation dynamics modulating pathways. For example, this can be accomplished by forming a cellular pathway modulator and ligand-contacted cell. A cell with a known cellular target is (i) contacted with a cellular pathway modulator that interacts with the cellular target and (ii) contacted with a ligand specific for the known cellular target. The contacted cell is then exposed to an enzyme condensation modulating agent and the response is then assayed. This allows one to determine the regulatory ability of the cellular pathway modulator on the state of enzyme condensation. The ability of the cellular pathway modulator to regulate enzyme condensation dynamics indicates that the pathway is an enzyme condensation dynamics modulating pathway.

The cellular target specific ligand is typically an agonist. The ligand can be a ligand specific for the cellular target, or can be a molecule that mimics the ligand and activates the cellular target pathway.

The cellular pathway modulator may be interference RNA or a kinase inhibitor and can affect different stages of the pathway. For example, some modulators may affect the earlier stages of a pathway, while others may affect the later stages. If a pathway splits into multiple pathways in the later stages, it can be useful to use cellular pathway modulators specific to each pathway.

In some instances, a control is used to compare the effects of the cellular pathway modulator on regulating enzyme condensation dynamics. For example, a cell contacted with a cellular pathway modulator, a ligand, and an enzyme condensation modulating agent may be compared to a control cell that has been contacted with a ligand and enzyme condensation modulating agent.

Yet another example of determining enzyme activity is by identifying agents that regulate enzyme condensation dynamics. This can be done by providing a cell, forming a molecule-contacted cell by contacting the cell with a molecule, exposing the molecule-contacted cell with an enzyme condensation promoting agent, exposing the molecule-contacted cell with an enzyme condensation disrupting agent, monitoring the state of enzyme condensation of the molecule-contacted cell after exposure to the enzyme condensation promoting agent and the enzyme condensation disrupting agent, and determining the molecule's ability to modulate the state of enzyme condensation. The step of monitoring the state of enzyme condensation can be performed with a label-free biosensor cellular assay or with fluorescent imaging.

In some circumstances, the enzyme condensation promoting agent can contact the cell before the enzyme condensation disrupting agent, while in others, the enzyme condensation disrupting agent contacts the cell before the enzyme condensation promoting agent. In either case, the time between contact of the promoting agent and the disrupting agent can be in the range from about 1 to about 60 minutes, and ranges there between. The time between cell contacts of the agents can be from about 1 to about 24 hours, and ranges there between.

Another aspect of this invention provides a method for classifying cellular targets, modulating pathways, and regulating agents based on their regulation of enzyme condensation dynamics. Classification can be based on correlation analysis, wherein the correlation between the enzyme condensation disrupting agent response and the enzyme condensation promoting agent response are determined. Classification can also be based on a similarity analysis, wherein the Hierarchy Euclidean clustering of the enzyme condensation disrupting agent's response and the enzyme condensation promoting agent's response are compared. Correlation or similarity analysis further includes examining the fluorescent patter of a fluorescent enzyme that is part of the enzyme condensation complex, wherein the fluorescent enzyme is introduced inside the cell through gene transfection and expression or through protein delivery.

The effects of the cellular target or the regulating agents on the cell's response to the enzyme condensation promoting agent or the enzyme condensation disrupting agent may vary. For example, the cellular target or the regulating agent may potentiate a promoting agent's response, but have little or no effect on (or even suppress) the disrupting agent's response, or vice versa. In other instances, the cellular target or regulating agent may suppress the promoting agent's or the disrupting agent's response. They may have no effect at all on either enzyme condensation agent, or may only potentiate one agent, but not the other.

By identifying and classifying which enzymes are active in different metabolic processes, it is possible to determine which enzymes are active in disease situations. Thus, another embodiment of the present invention provides for the treatment of a disease that is pathophysiologically related to enzyme condensation, such as a metabolic disorder, cancer, or an inflammatory disease. The method includes administering a therapeutically effective amount of an enzyme condensation dynamics modulator. A therapeutically effective amount can range from 1-100 micromol/kg of an enzyme condensation dynamics modulator. Additionally, a therapeutic agent can be administered with the enzyme condensation dynamics modulator, via the same or different routes, and work either synergistically or in combination with the dynamics modulator. In some cases, a therapeutic agent may need to be administered after the enzyme condensation dynamics modulator to have an effect on the disease.

The enzyme condensation dynamics modulator and the therapeutic agent can be administered in the range from about 1 to 60 minutes apart, and ranges there between or from about 2 to about 72 hours, and ranges there between.

### EXAMPLE

The invention is further illustrated by the following example. The example is provided for illustrative purposes only, and is not to be construed as limiting the scope or content of the invention in any way.

Enzymatic activity of the MurA enzyme in Bacillus subtilis was measured using Fosfomycin or Vancomycin. Exponentially growing cells expressing MurA-CFP under its natural promoter were stained with FM4-64 (1 μg ml⁻¹). After one hour in exponential growth, 1 ml of culture was centrifuged. The supernatant was collected and added to 2 ml of melted 3.5% agarose solution in LB. The resulting 1.2% solution of molten agar/culture supernatant was supplemented with 0.5 ig ml⁻¹ FM 4-64 and 5 mM Fosfomycin or 0.5 μg ml⁻¹ Vancomycin, added to the well of a culture slide (wells 18 mm diameter×1.75 mm depth) and covered with a glass slide.

After cooling, the cover glass was removed and two air pockets cut out of the agarose with a 15 ml tube, leaving a 3-5 mm agar bridge in the center of the well. Seven microliters of the remaining culture was spread over the agar, partially dried and sealed with cover glass. After cooling, the slide was removed and two air pockets were cut out of the agar leaving a 3-5 mm agar bridge in the center of the well. Cells suspended in LB with FM4-64 (0.5 μg ml⁻¹) were added at the agar bridge and covered by a glass coverslip. To prevent drying during the experiment, 50% glycerol was applied to the region of contact between the slide and the coverslip.

The slide equilibrated in an environmentally controlled chamber at 30° C. (Precision Control Weather Station) for at least 10 min prior to visualization. Images were acquired every 5 minutes for 4 hours, using an Applied Precision Spectris microscope, with a 100× objective using phase contrast and captured by a Hamamatsu Orca-ER camera using Nikon Elements BR software. CFP and TRITC (FM4-64) exposures were 400 ms. Fosfomycin and Vancomycin were obtained from Sigma, cerulenin was obtained from Cayman Chemical, and FM4-64 to stain the cellular membranes was from Invitrogen. After 2 hours of imaging MurA-CFP expressing cells stopped growing but the condensation of the enzyme was detected as shown by a bright fluorescent spot.

Thus, from the foregoing disclosure and detailed description of certain preferred embodiments for methods for identifying enzyme activity through determination of its localization, and their uses for treating pathophysiological disorders, it is apparent that various modifications, additions, and other alternative embodiments are possible without departing from the scope of the present invention. The embodiments described herein were chosen to provide the best illustration of the present invention, and thus one skilled in the art can practice the invention in ways other than the described embodiments. The present invention is only limited by the claims which follow.

What is claimed is:

1. A method of identifying cellular targets involved in regulating enzyme condensation dynamics, the method comprising the steps of:
providing a cell that has a cellular target;
contacting the cell with a molecule that interacts with the cellular target to form a molecule-contacted cell;
sequentially exposing the molecule-contacted cell to an enzyme condensation promoting agent and an enzyme condensation disrupting agent;
monitoring the state of enzyme condensation of the molecule-contacted cell after exposure to the enzyme condensation promoting agent and the enzyme condensation disrupting agent; and
determining the molecule's ability to modulate the state of enzyme condensation thereby identifying cellular targets involved in regulating enzyme condensation dynamics.

2. The method of claim 1, wherein the molecule that interacts with the cellular target is selected from the group consisting of:
signaling effectors, hormones, antibodies, antibiotics, amino acids, carbohydrates, carboxylic acids and combinations thereof.

3. The method of claim 1, wherein the enzyme condensation promoting agent is selected from the group consisting of:
insulin, EGF, glucose, and 1-methionine.

4. The method of claim 1, wherein the enzyme condensation disrupting agent is selected from the group consisting of:
anti-her2, fosfomycin, and rapamycin.

5. The method of claim 1, wherein the cellular targets are classified based on similarity analysis or correlation analysis.

6. The method of claim 1, wherein the monitoring the state of enzyme condensation of the molecule-contacted cell is carried out by fluorescence imaging or whole cell sensing.

7. A method of identifying an enzyme condensation dynamics modulating pathway, the method comprising the steps of:
providing a cell that has a cellular target;
contacting the cell and (i) a cellular pathway modulator that interacts with the cellular target and (ii) a cellular target specific ligand thereby forming a cellular pathway modulator ligand-contacted cell;
exposing the cellular pathway modulator ligand-contacted cell to an enzyme condensation modulating agent;
assaying the cell response to the enzyme condensation modulating agent; and
determining the cellular pathway modulator's ability to regulate the state of enzyme condensation, thereby identifying enzyme condensation dynamics modulating pathways.

8. The method of claim 7, wherein the enzyme condensation modulating agent is an enzyme condensation disrupting agent selected from a group consisting of:
anti-her2, fosfomycin, and bacterial products.

9. The method of claim 7, wherein the ligand is an agonist.

10. A method of identifying agents that regulate enzyme condensation dynamics, the method comprising the steps of:
providing a cell that has a cellular target;
forming a molecule-contacted cell by contacting the cell and a molecule that interacts with the cellular target;
sequentially exposing the molecule-contacted cell with an enzyme condensation promoting agent and an enzyme condensation disrupting agent;
monitoring the state of enzyme condensation of the molecule-contacted cell after exposure to the enzyme condensation promoting agent and the enzyme condensation disrupting agent; and
determining the molecule's ability to modulate the state of enzyme condensation, thereby identifying agents that regulate enzyme condensation dynamics.

11. The method of claim 10, wherein the molecule is selected from the group consisting of:
signaling effectors, hormones, antibodies, antibiotics, amino acids, carbohydrates, carboxylic acids, and combinations thereof.

12. The method of claim 10, wherein the enzyme condensation promoting agent is selected from the group consisting of:
insulin, EGF, Glucose, and 1-methionine.

13. The method of claim 10, wherein the enzyme condensation disrupting agent is selected from the group consisting of:
anti-her2, fosfomycin, and rapamycin.

14. The method of claim 10, wherein the enzyme condensation dynamics modulator is classified based on similarity analysis.

15. The method of claim 10, wherein the monitoring the state of enzyme condensation of the molecule-contacted cell is carried out by fluorescence imaging or whole cell sensing.

16. A method of determining metabolic state of an enzyme through enzyme condensation, the method comprising the steps of:
providing a cell having a cellular target;
attaching a reporter protein to the cellular target;
sequentially exposing the cell to an enzyme condensation promoting agent and an enzyme condensation disrupting agent;
monitoring the state of enzyme condensation of the cell after exposure to the enzyme condensation promoting agent and the enzyme condensation disrupting agent; and
determining the state of enzyme condensation.

17. A method of treating a subject having a disease that is pathophysiologically related to enzyme condensation, the method comprising the step of:
administering a therapeutically effective amount of an enzyme condensation dynamics modulator for modulating the enzyme condensation to mitigate the disease.
